(19) United States
(12) Patent Application Publication
(10) Pub. No.: US 2007/0250145 A1
(43) Pub. Date: Oct. 25, 2007

(54) DEVICE FOR THE TRANSDERMAL STIMULATION OF A NERVE OF THE HUMAN BODY

(75) Inventors: Thomas KRAUS, Nuernberg (DE); Stefan DIETRICH, Erlangen (DE)

Correspondence Address: LUCAS & MERCANTI, LLP, 475 PARK AVENUE SOUTH, 15TH FLOOR, NEW YORK, NY, 10016, US

(73) Assignee: CERBOMED GMBH, Erlangen (DE)

(21) Appl. No.: 11/749,924

(22) Filed: May 17, 2007

Related U.S. Application Data
(63) Continuation of application No. PCT/EP06/00513, filed on Jan. 21, 2006.

(30) Foreign Application Priority Data
Jan. 26, 2005 (DE) 10 2005 003 735.6

(51) Int. Cl. A61N 1/36

(52) U.S. Cl. 607/136; 607/2

(57) ABSTRACT

The invention relates to a device (1) for transcutaneous stimulation of a nerve of the human body, which device (1) comprises at least one stimulation electrode (2) and at least one reference electrode (3) for transcutaneous nerve stimulation, the at least one stimulation electrode (2) and the at least one reference electrode (3) being connected to a control unit (4) and being able to be supplied with an electrical current from the latter, and the at least one stimulation electrode (2) and the at least one reference electrode (3) being arranged in or on a housing (5) which is designed to be fitted on or in the human ear. To make the nerve stimulation effective and to make it easier to manage for the patient, it is proposed, according to the invention, that the housing (5) has a bow-shaped extension piece (6) designed to be inserted into the auditory canal, said bow-shaped extension piece (6) matching the shape of the entrance to the auditory canal or of the external auditory canal, and with an electrode head (7) which is arranged at the end of the bow-shaped extension piece (6) and which has two contact points (8, 9) for the two electrodes (2, 3).

The invention relates to a device for transcutaneous stimulation of a nerve of the human body, which device comprises at least one stimulation electrode and at least one reference electrode for transcutaneous nerve stimulation, the at least one stimulation electrode and the at least one reference electrode being connected to a control unit and being able to be supplied with an electrical current from the latter, and the at least one stimulation electrode and the at least one reference electrode being arranged in or on a housing which is designed to be fitted on or in the human ear.

Devices of the type in question are known in many forms. For example, transcutaneous nerve and muscle stimulation is used in sports medicine for stimulating a muscle, for which purpose electrodes are placed or affixed on the skin. An advantage of this is that the surface of the skin is not damaged when the electrode is placed in position (non-invasive application of the electrode). By contrast, previously known possibilities also exist in which electrodes are implanted into or under the skin.

In the devices for transcutaneous nerve stimulation, it is known, by means of invasive or non-invasive electrical stimulation of the nerves, to influence their neuroelectrical quality and thus to influence the function of the nerves that are to be stimulated. The aim of this approach is to incite psychovegetative changes, for example stress relief, or to treat neuro-sychiatric disturbances.

For many years, particular importance has been attached to stimulation of the vagus nerve. As the tenth cranial nerve, it is the main nerve of the parasympathetic system. It is also involved in the motor control of the larynx and pharynx and transmits taste sensations from the base of the tongue and sensations of touch from the pharynx, larynx and part of the external auditory canal (auricular branch).

Invasive stimulation of the vagus nerve in particular has in the meantime become an established therapeutic procedure in neurology for treatment of epilepsy, in which connection reference is made to Penry JK, Dean JC: Prevention of intractable partial seizures by intermittent vagal stimulation in humans: preliminary results. Epilepsia 1990; 31 Suppl 2: 40-43, and to Uthman B M, Wilder B J, Hammond E J, Reid S A: Efficacy and safety of vagus nerve stimulation in patients with complex partial seizures. Epilepsia 1990; 31 Suppl 2: 44-50.

In the above, the patient's vagus nerve is exposed on the left region of the neck by neurosurgery and a current conductor is wound around it as an electrode. The device for generating current impulses is implanted under the skin in the left shoulder area. The vagus nerve stimulator can later be programmed from outside by means of an electromagnetic field. Electrical excitation of the vagus nerve causes a stimulation of the brain in various areas, as can be demonstrated by imaging methods. In addition to its effectiveness in epilepsy, stimulation treatment also has psychological effects, for example antidepressive effects, in which connection reference may be made to Elger G, Hoppe C, Falkai P, Rush A J, Elger C E: Vagus nerve stimulation is associated with mood improvements in epilepsy patients. Epilepsy Res 2000; 42:203-210.

For this reason, the method has in recent times also been used in psychiatry and has proven useful in the management of otherwise treatment-resistant forms of severe depression (see in this connection Carpenter L L, Friehs G M, Price L H: Cervical vagus nerve stimulation for treatment-resistant depression. Neurosurg Clin N Am 2003; 14:275-282, Goodnick P J, Rush A J, George M S, Marangell L B, Sackeim H A: Vagus nerve stimulation in depression. Expert Opin Pharmacother 2001; 2:1061-1063, and Rush A J, George M S, Sackeim H A, Marangell L B, Husain M M, Giller C, Nahas Z, Haines S, Simpson R K, Jr., Goodman R: Vagus nerve stimulation (VNS) for treatment-resistant depressions: a multicenter study. Biol Psychiatry 2000; 47: 276-286).

For stimulation of nerves in general, various approaches have already been proposed.

U.S. 5,458,625 discloses a device of the type mentioned at the outset for nerve stimulation by means of electrical impulses. The current impulses are introduced by means of electrodes that are fitted on the earlobe of the patient.

Other solutions for introducing electrical impulses into the human body are known from JP 101 08 913 A, from DE 39 18 329 A1 and from FR 2 717 699 A1.

EP 0 645 162 B1 describes a stimulation device for stimulation of muscles and nerves, which device comprises a function generator for generating a wave shape with a sequence of pulses. It is intended particularly for treatment of pain. EP 0 757 573 A1 and EP 1 064 047 A1 describe systems and methods for electrical nerve stimulation in general.

EP 1 145 736 A2 proposes an implantable, multi-mode neurostimulator. EP 0 972 538 A2 describes a system for delivering an electrical stimulus to part of the nervous system by means of a needle electrode. EP 1 048 319 A2 proposes a system for selective activation of brain neurons, spinal column parenchyma or peripheral nerves, which system works with an insertable cannula.

EP 1 022 034 A1 discloses a method and a device for stimulation of muscles or nerve tissue by generation of impulse signals. EP 1 393 773 A1 describes an external nerve stimulator for stimulating the phrenic nerve by means of an oesophageal electrode. EP 0 962 234 A1describes a device for electrical nerve stimulation, but does not disclose the placement of the electrodes.

WO 97/45160 describes a device for modulation of the neuronal brain plasticity. WO 01/00273 describes a non-invasive method and device for stabilizing the heart rate by means of skin electrodes. EP 1 420 854 A2 uses a diaphragm electrode for treatment of neuropsychiatric disturbances. Finally, EP 1 418 981 A1 involves stimulating nerves in the vicinity of the diaphragm for treatment of movement disorders.

The following circumstances have proven disadvantageous in the previously known solutions:

Many methods require invasive introduction of the stimulation electrode and, in addition to the usual risks posed by anaesthesia and a surgical intervention, they are associated in particular with the danger or nerve damage and the risk of infection.

If the treatment should prove unsuccessful, the stimulation device has to be removed again, with the same risks as described above.

There is therefore a limit to the acceptance on the art of patients to undergo such an operation.

In addition, there is sometimes a feeling of being at the mercy of a machine implanted in ones body and of not being easily able to get rid of it if necessary.

The described devices often also involve a large and rather unwieldy construction and are therefore suitable only for clinical use, not for long-term stimulation treatment at home.

The stimulation device and electrodes are often connected via quite long cables, which has a negative impact on their handling.

Because of the handling involved (e.g. the handling of large adhesive electrodes), stimulation can be carried out only when lying down, not inconspicuously while going about ones daily routine.

For wide-ranging therapeutic application of the method of transcutaneous vagus stimulation, it is desirable to integrate the technique into a small and manageable, and if possible wireless device which can easily be worn at all times in an inconspicuous manner.

The object of the invention is therefore to develop a device of the type mentioned at the outset in such a way that this aim is achieved. In other words, the object is to create a device for nerve stimulation which permits a particularly efficient and simple stimulation of the vagus nerve, specifically during ones daily routine and in a straightforward and virtually unnoticeable way. The device should be able to be used particularly easily by the patient and should be able in particular to be removed quickly from the body, if need be. The object is to propose an efficient external non-invasive device which is used to stimulate the vagus regions and which is distinguished by a high degree of wearing comfort, and in which the stimulation is intended to be able to take place at any given time and in an uncomplicated way. Moreover, the patient is to be afforded the possibility of monitoring the course of the therapy. In addition, the device is to be used for carrying out a simple, stable and safe stimulation method.

This object is achieved, according to the invention, by the fact that, for optimal positioning of the electrodes, the housing has a bow-shaped extension piece designed to be inserted into the auditory canal, said bow-shaped extension piece matching the shape of the entrance to the auditory canal or of the external auditory canal, and with an electrode head which is arranged at the end of the bow-shaped extension piece and which has two contact points for the two electrodes.

The device is designed and suitable for stimulation of the vagus nerve in the area of the external auditory canal and/or the auricle.

The control unit is preferably arranged in the housing. However, provision can also be made for the control unit to be removable from the housing and connected to the electrodes. The connection can in this case be a wired connection. However, a wireless connection is also possible, for example a radio connection.

The electrode head is advantageously made of a soft material, in particular of permanently soft silicone. The contact points can be formed by metal balls. They can also be formed by flat surface electrodes. It is also possible that the contact points are formed by an element made of a material with electrical surface conductivity, in particular of a sponge with graphite inserts.

The control unit is able to influence the frequency of an alternating current flowing through the electrodes. The same applies to influencing the level of the current flowing through the electrodes, to influencing the length of impulses of the current flowing through the electrodes, to influencing stimulation time intervals of the current flowing through the electrodes, and/or to influencing the time profile of the current flowing through the electrodes.

A rechargeable battery is preferably arranged in the device and supplies current to the control unit.

Provision can also be made for the device to comprise a sensor for measuring a physiological parameter of the patient. This parameter can, for example, be the patient's pulse or the oxygen saturation of the patient's blood. A memory chip can also be provided for storing the data measured by means of the sensor.

The electrodes can be integrated into the earpiece, or into the headset of a hands-free mobile telephone unit, and the control unit can be integrated into a mobile telephone. Provision can be made for the connection between electrodes and control unit to be established via a radio connection, in particular via a Bluetooth connection or a WLAN connection.

It is also possible for the electrodes to be integrated into the headphones of a music playback system, and for the control unit to be integrated into the music playback system.

Since the vagus nerve also has afferent paths in the skin of the external auditory canal, electrical stimulation of the vagus nerve is also possible through the skin of the ear and thus non-invasively by means of a transcutaneous electrode. It has already been successfully demonstrated that electrical stimulation of the vagus nerve via afferent pathways in the external auditory canal leads to a derivable potential on the surface of the skull (sensory evoked potential).

The proposed concept thus stimulates the nerve branches (auricular branch) of the vagus nerve in the area of the external auditory canal and thus influences its function. This is achieved by integrating the technology of transcutaneous vagus nerve stimulation into a stimulation device which is to be worn on or behind the ear and whose outward appearance is similar to that of a hearing aid.

External (non-invasive) stimulation units for the vagus nerve in the ear region do not yet exist. The invention remedies this situation. The previously known non-invasive nerve stimulation methods by means of application of current make use of peripheral nerve and muscle stimulation for treatment of pain (transcutaneous electrical nerve stimulation—TENS), muscle training (electrical muscle stimulation—EMS) or electroacupuncture of defined meridian points. None of these methods is intended for stimulating the vagus nerve in the ear region in order to bring about changes in the central nervous system.

By contrast, the invention is concerned with the transcutaneous stimulation of the vagus nerve in the ear region and for this purpose proposes a device that is particularly easy to use.

With the proposal according to the invention, a transcutaneous stimulation of the vagus nerve is therefore possible, particularly for the treatment of neuropsychiatric disturbances, in which a stimulation electrode placed in or on the external auditory canal is provided for transcutaneous stimulation of the auricular branch of the vagus nerve, and a reference electrode is placed in or on the external auditory canal, these electrodes preferably being connected to a control unit which is worn on or behind the ear.

When the earpiece is in use, the electrodes touch the skin surface of the external auditory canal and are therefore able to stimulate the vagus nerve areas located there.

An illustrative embodiment of the invention is shown in the drawing, in which:

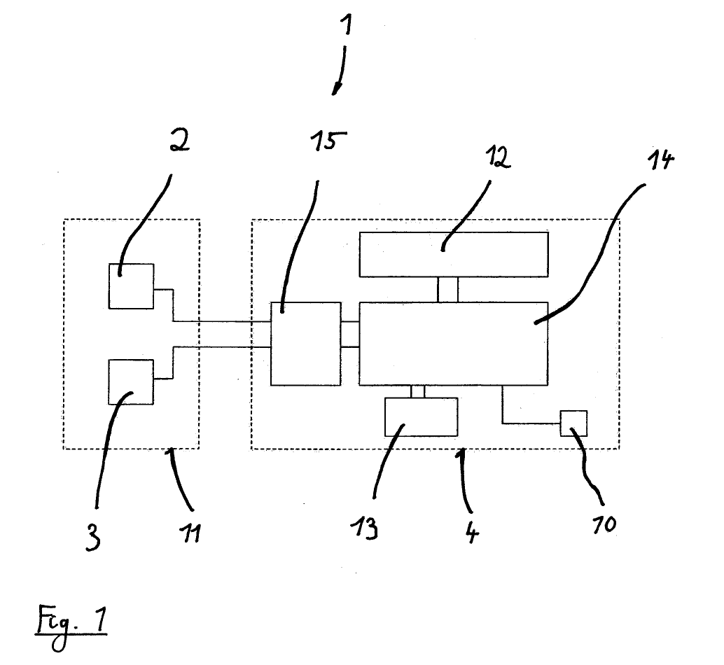

FIG. 1 is a schematic circuit diagram of a device for transcutaneous stimulation of the auricular branch of the vagus nerve, and

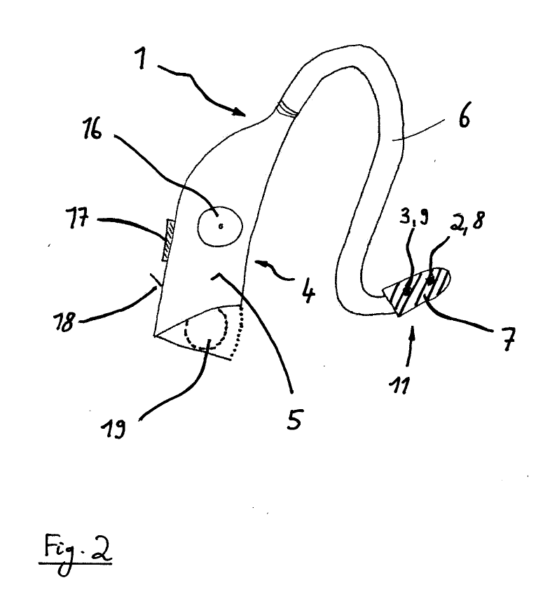

FIG. 2 shows the stimulation device, designed as a behind-the-ear device.

The circuit diagram of a device 1 for transcutaneous stimulation of the vagus nerve is shown schematically in FIG. 1. The auricular branch in particular is stimulated in order to influence psychovegetative parameters. In this way, for example, stress levels can be reduced, or a positive influence can be exerted on depressions or other neuropsychiatric disturbances.

The device 1 is composed principally of the stimulation electrode unit 11 (indicated with broken lines on the left-hand side of FIG. 1) and of the control unit 4 (indicated with broken lines on the right-hand side of FIG. 1).

The stimulation of the nerve takes place via the stimulation electrode 2. The reference electrode 3 serves as an electrical reference point. Both electrodes 2, 3 form the stimulation electrode unit 11. Electrodes 2 and 3 for transcutaneous stimulation are known, commercially available and easy to produce.

The stimulation frequency and the stimulation strength are predetermined and generated by the control unit 4. These parameters are set by various control elements 12. Oscillating signals are needed for transcutaneous stimulation. They are generated by an oscillator 13 located in the control unit 4. The input and output signals that are delivered via an input/output circuit 15 of the stimulation electrode unit 11 are processed in a logic and control circuit 14. The current is supplied from a battery 10.

As can be seen from FIG. 2, the device 1 is similar in structure to a behind-the-ear hearing aid and has a housing 5. The stimulation electrode unit in the form of an electrode head or an ear electrode 7 is inserted into the external auditory canal, such that the stimulation electrode 2 and the reference electrode 3 come to lie on the skin surface. The connection between the electrode head 7 and the part of the housing 5 shown on the left-hand side of FIG. 2 is designed as a bow-shaped extension piece 6, through which all the input and output lines between stimulation electrode unit and control unit are also routed; the bow-shaped extension piece 6 is fitted over the upper margin of the auricle. At the end of the connection or link, the control unit 4 is located in the housing 5 with an approximate size of 5 cm×2 cm×1 cm.

Integrated into the control unit 4 there is, in the first instance, a stimulation strength regulator 16 for regulating the amplitude (strength) of the stimulation signal. High amplitudes stimulate the nerve more than low amplitudes. Moreover, the required stimulation strength varies between individuals.

The control unit 4 also contains a stimulation frequency regulator 17 for regulating the frequency pattern of the stimulation signal. Thus, signals following one another in rapid succession can be controlled just as can signals that follow one another at a greater interval.

An on/off switch 18 is also provided for activating and deactivating the device 1. A battery compartment 19 is used to accommodate a small button-cell battery, preferably of size 13 to 675.

One example of the action of the proposed device on the vagus nerve is the following: The applied current is between 0.25 and 1.5 mA. The frequency of the current is between 20 and 30 Hz. The pulse width is between 250 and 500 μs. The current is applied every 3 to 5 minutes for ca. 30 seconds.

The proposed stimulation device 1 is very small and is therefore eminently suitable for home use. It affords the wearer great freedom, because its placement behind the ear is very advantageous and discrete.

The stimulation and reference electrodes 2, 3 must have electrical contact with the surface of the patient's skin, and this contact is permitted by contact points 8, 9 which can be designed as small metal balls. The electrodes 2, 3 lie on the inner face of the tragus, i.e. an anatomical part of the auricle. The distance between the contact points 8, 9 is preferably between 1 mm and 15 mm, particularly preferably between 2 mm and 6 mm.

In another variant of the solution, the earpiece can be inserted farther into the auditory canal and can there also provide stimulation of the vagus nerve. For this purpose, the electrodes 2, 3 can be designed as flat surface electrodes, for example. Further nerve endings of the vagus nerve are stimulated deeper within the auditory canal.

The electrodes 2, 3 are connected to cables (not shown) which are routed in a concealed manner within the earpiece. The cable connections in turn are connected to the control unit 4 preferably located behind the ear. The connection is established via the bow-shaped extension piece 6, as has been explained. The stimulation frequency, stimulation strength, impulse duration, stimulation intervals and current form are set via the stimulation frequency regulator 17.

In a similar way to an in-the-ear hearing aid, the whole technology can also be integrated into a device that comes to lie in the concha of the ear and fills it.

The device is supplied with current by the battery 10 and is therefore independent of an external power source. Provision can be made for the current to be supplied via a rechargeable battery 10 which is integrated into the housing 5. For the recharging operation, the device 1 is inserted into a small specially designed case which is connected to an external power source and which charges the battery 10 overnight by induction, for example.

The earpiece can additionally be provided with a sensor for measuring the pulse and oxygen saturation. Such sensors are known for measurement of respiratory function and pulse and are commercially available. The measured values can be recorded on a memory chip located in the housing 5 behind or in the ear, such that they can later be read out by a physician via a cableless interface and can be evaluated using software. From the change in the pulse rate variability calculated by the software, the physician is able to obtain important information concerning the psychovegetative modulation effect of the stimulation device and is thus also provided with control data over the course of the therapy.

The described device can be constructed according to standard values, or the earpiece and other parts can be manufactured individually.

In an alternative embodiment, the electrode head 7 and the control unit 4 are stored separately and are connected via a cable.

In a further alternative, the stimulation technology can be integrated into a mobile telephone and into its hands-free unit. The control unit 4 and its electronics can in this case be integrated into the circuitry of the mobile telephone. The stimulation unit 7 with stimulation electrode 2 and reference electrode 3 can be installed in the earpiece of the hands-free unit. The communication between earpiece and mobile telephone can be wireless, for example by means of Bluetooth technology, or can be via a connecting cable.

It is also possible for the technology to be integrated into headphones and devices for example for digital media playback. These can be MP3 players or, in particular, MD players or Discmans.

### LIST OF REFERENCE NUMBERS

1 device for transcutaneous stimulation of a nerve
2 stimulation electrode
3 reference electrode
4 control unit
5 housing
6 bow-shaped extension piece
7 electrode head
8 contact point
9 contact point
10 battery
11 stimulation electrode unit
12 control elements
13 oscillator
14 logic and control circuit
15 input/output circuit
16 stimulation strength regulator
17 stimulation frequency regulator
18 on/off switch
19 battery compartment

1. Device for transcutaneous stimulation of a nerve of the human body, which device comprises at least one stimulation electrode and at least one reference electrode for transcutaneous nerve stimulation, the at least one stimulation electrode and the at least one reference electrode being connected to a control unit and being able to be supplied with an electrical current from the latter, and the at least one stimulation electrode and the at least one reference electrode being arranged in or on a housing which is designed to be fitted on or in the human ear, wherein the housing has a bow-shaped extension piece designed to be inserted into the auditory canal, said bow-shaped extension piece matching the shape of the entrance to the auditory canal or of the external auditory canal, and with an electrode head which is arranged at the end of the bow-shaped extension piece and which has two contact points for the two electrodes.

2. Device according to claim 1, wherein the device is designed and suitable for stimulation of the vagus nerve in the area of the external auditory canal and/or the auricle.

3. Device according to claim 1, wherein the control unit is arranged in the housing.

4. Device according to claim 1 wherein the control unit is removable from the housing and is connected to the electrodes.

5. Device according to claim 4, wherein the connection is a wired connection.

6. Device according to claim 4, wherein the connection is wireless, and in particular is a radio connection.

7. Device according to claim 1, wherein the electrode head is made of a soft material, in particular of permanently soft silicone.

8. Device according to claim 1, wherein the contact points are formed by metal balls.

9. Device according to claim 1, wherein the contact points are formed by flat surface electrodes.

10. Device according to claim 1, wherein the contact points are formed by an element made of a material with electrical surface conductivity, in particular of a sponge with graphite inserts.

11. Device according to claim 1, wherein the control unit is able to influence the frequency of an alternating current flowing through the electrodes.

12. Device according to claim 1, wherein the control unit is able to influence the level of the current flowing through the electrodes.

13. Device according to claim 1, wherein the control unit is able to influence the length of impulses of the current flowing through the electrodes.

14. Device according to claim 1, wherein the control unit is able to influence the stimulation time intervals of the current flowing through the electrodes.

15. Device according to claim 1, wherein the control unit is able to influence the time profile of the current flowing through the electrodes.

16. Device according to claim 1, wherein a rechargeable battery is arranged in the device and supplies current to the control unit.

17. Device according to claim 1, wherein it comprises a sensor for measuring a physiological parameter of the patient.

18. Device according to claim 17, wherein the physiological parameter is the patient's pulse.

19. Device according to claim 17, wherein the physiological parameter is the oxygen saturation of the patient's blood.

20. Device according to claim 17, further comprising a memory chip for storing the data measured by means of the sensor.

21. Device according to claim 1, wherein the electrodes are integrated into the earpiece, or into the headset of a hands-free mobile telephone unit, and in that the control unit is integrated into a mobile telephone.

22. Device according to claim 21, wherein the connection between electrodes and control unit is established via a radio connection, in particular via a Bluetooth connection or a WLAN connection.

23. Device according to claim 1, wherein the electrodes are integrated into the headphones of a music playback system, and in that the control unit is integrated into the music playback system.
